(19) United States
(12) Patent Application Publication
(10) Pub. No.: US 2023/0028772 A1
(43) Pub. Date: Jan. 26, 2023

(54) MICROPARTICLE FOR DRUG LOADING, DRUG LOADING MICROPARTICLE, PARTICLE CONTAINING TUBE, AND IMPLANTATION SYSTEM FOR MICROPARTICLE

(71) Applicant: SUZHOU YIBEN LIFE TECHNOLOGY CO., LTD, Suzhou (CN)

(72) Inventors: Yonghua DONG, Suzhou (CN); Yuan HE, Suzhou (CN)

(73) Assignee: SUZHOU YIBEN LIFE TECHNOLOGY CO., LTD, Suzhou (CN)

(21) Appl. No.: 17/933,822

(22) Filed: Sep. 20, 2022

Related U.S. Application Data
(63) Continuation-in-part of application No. PCT/CN2021/081950, filed on Mar. 20, 2021.

(30) Foreign Application Priority Data
Mar. 20, 2020 (CN) 202010199438.2
Mar. 20, 2020 (CN) 202010199724.9

(51) Int. Cl. A61K 9/00; A61K 9/50; A61M 5/00; A61N 5/10

(52) U.S. Cl. CPC A61K 9/0024; A61K 9/50; A61M 5/007; A61N 5/1027

(57) ABSTRACT

A microparticle for drug loading, a drug loading microparticle, a particle containing tube, and an implantation system for the microparticle. The microparticle for drug loading includes a housing (31) and a drug loading part (34) located inside the housing and is used for being implanted into body tissues by means of a puncture needle (5); the housing (31) is provided with at least one micro-hole (33) running through the wall thickness of the housing (31); and the drug loading part (34) is located inside the housing (31) and is used for loading drugs. The microparticle for drug loading/drug loading microparticle can achieve different types of drug loading and different release speeds, can be directly implanted into tissues, and have the technical advantages of both microspheres and radioactive particles.

### BACKGROUND

### Technical Field

The present disclosure relates to a microparticle for drug loading, a drug loading microparticle, a particle containing tube for holding the microparticle for drug loading/drug loading microparticle and an implantation system for implanting the drug loading microparticle, and belongs to the technical field of medical interventional devices.

### Related Art

With the popularization of interventional procedures, microsphere or microcapsule interventional techniques are gaining popularity. Currently, drug loading microspheres can be divided into biodegradable and non-biodegradable types. Polylactic acid (PLA, also known as polylactide), has been approved by the U.S. FDA as a medical polymer. Microspheres made of PLA have been widely used as carriers of peptides and protein-based drugs in many fields such as immunotherapy, gene therapy, tumor therapy, and orthopedic repair.

Typically, microspheres or microcapsules are delivered into the blood vessel and driven by blood to travel to a plurality of locations in the blood vessel, where they are not completely fixed. It is well known that when the tumor diameter increases to 2 mm or more, there need to be new capillaries to supply blood, so that angiogenesis plays an important role in tumor spread. In tumor therapy, microspheres are delivered to tumor tissues through microcatheters via the tumor-feeding artery pathway and are mainly used in the treatment of tumors or diseased tissues with rich blood supply. For tumor tissues that are not rich in tumor blood supply, or where the tumor blood supply has been changed to a fine and multi-branched collateral vascular supply after multiple interventions, the delivery of microspheres via vascular access is limited or fails. In addition, various arteriovenous fistulas often remain in the tumor, while the microspheres delivered via the arterial route not only fail to stay in the tumor as designed to play a therapeutic role, but also leak out through the fistula into the vein and eventually reach the lung, leading to serious consequences.

The implantation of radioactive particles has become one of the common means of treating cancer. However, the radioactive particles themselves in the prior art do not allow for different drug loading.

### SUMMARY

The primary technical problem to be solved by the present disclosure is to provide a microparticle for drug loading.

Another technical problem to be solved by the present disclosure is to provide a drug loading microparticle.

A further technical problem to be solved by the present disclosure is to provide a particle containing tube for holding the microparticle for drug loading/drug loading microparticle and an implantation system for implanting the drug loading microparticle.

In order to achieve the above purposes, the following technical solutions are used by the present disclosure.

According to a first aspect of an embodiment of the present disclosure, provided is a microparticle for drug loading used for being implanted into body tissues by means of a puncture needle including a housing and a drug loading part located inside the housing, the housing being provided with at least one micro-hole running through the wall thickness of the housing;

and the drug loading part being located inside the housing and being used for loading drugs.

Preferably, the housing is a biodegradable material and a specific surface area of the micro-hole of the housing varies with the degradation time.

Preferably, there are a plurality of micro-holes and at least one of the plurality of micro-holes does not run through the wall thickness of the housing; or the micro-hole running through the wall thickness of the housing is filled with a slow-dissolving hole filler.

Preferably, the micro-hole has a predetermined number, size, location or area that varies in the wall thickness direction.

Preferably, the drug loading part is a hollow area; or the drug loading part is a solid having a different material than the housing and the solid can adsorb liquid entering the housing through the micro-hole.

According to a second aspect of an embodiment of the present disclosure, provided is a drug loading microparticle used for being implanted into body tissues by means of a puncture needle including a housing and a drug loading part located inside the housing, the housing being provided with at least one micro-hole running through the wall thickness of the housing;

the drug loading part being located inside the housing and having loaded contents; and

the contents being biocompatible and dissolvable into the body tissues outside the housing.

Preferably, the housing is provided with at least one micro-hole which does not run through the wall thickness of the housing or at least one micro-hole which is filled with a slow-dissolving hole filler.

Preferably, the housing of the drug loading microparticle loads a drug and/or a contrast agent, the drug being a drug different from that in the drug loading part.

According to a third aspect of an embodiment of the present disclosure, provided is a particle containing tube for holding a plurality of microparticles for drug loading/drug loading microparticles.

Preferably, each of the drug loading parts in the microparticles for drug loading/drug loading microparticles holds a different content.

Preferably, at least two housings of the microparticles for drug loading/drug loading microparticles have different specific surface areas of micro-holes.

According to a fourth aspect of an embodiment of the present disclosure, provided is an implantation system for implanting the drug loading microparticle, including a puncture needle or a catheter, and further including the particle containing tube as described above.

Preferably, the particle containing tube has a plurality of drug loading microparticles, where at least two of the drug loading microparticles contain different contents.

Preferably, the last one in the plurality of drug loading microparticles loads a procoagulant, a contrast agent or a radioactive particle.

The microparticles for drug loading/drug loading microparticles (microparticles for short) provided by the present disclosure can achieve different types of drug loading and different release speeds, can be directly implanted into tissues, and have the technical advantages of both microspheres and radioactive particles. In addition, the microparticles provided by the present disclosure can achieve different microparticles with different drug release speed profiles through the integrated design such as a specific surface area of the micro-hole and filler. The precise control of drug release can be achieved by implanting a plurality of microparticles with different drug release speed profiles at a time. Further, since the microparticles have different drug loadings, the microparticles with different drugs can be implanted at a time, thus allowing different drugs to promote each other and improve efficacy.

### BRIEF DESCRIPTION OF THE DRAWINGS

FIG. 1 is a schematic diagram showing the structure of microparticles for drug loading in an embodiment of the present disclosure;

FIG. 2 is a schematic diagram showing the structure of a micro-hole in a housing of the microparticles for drug loading in FIG. 1;

FIG. 3 is a schematic diagram showing the process for manufacturing the microparticles for drug loading in FIG. 1;

FIG. 4 is a schematic diagram showing the shape of a catheter and gelatin particles after freeze-drying in FIG. 3;

FIGS. 5A to 5D are schematic diagrams showing the drug loading steps of drug loading microparticles provided by an embodiment of the present disclosure;

FIG. 6 is a schematic diagram showing the use of a puncture needle to push the microparticles in FIG. 1 into the body;

FIG. 7A is a schematic diagram showing a particle containing tube provided by an embodiment of the present disclosure;

FIG. 7B is a schematic diagram showing another particle containing tube provided by an embodiment of the present disclosure;

FIG. 8 is a schematic diagram showing the release speed profile of the drug loaded in the microparticles for drug loading in an embodiment of the present disclosure;

FIG. 9 is a schematic diagram showing the drug loading capacity of the microparticles for drug loading in an embodiment of the present disclosure;

FIG. 10 is a diagram showing the release profile of the drug loaded in the microparticles for drug loading in an embodiment of the present disclosure;

FIG. 11 is a schematic diagram showing implantation of drug loading microparticles using a puncture needle in an embodiment of the present disclosure;

FIG. 12 is a schematic diagram showing the tumor suppression effect of the drug loading microparticles in an embodiment of the present disclosure; and

FIG. 13 is a diagram showing the appearance of the drug loading microparticles in an embodiment of the present disclosure.

### DETAILED DESCRIPTION

The following is a detailed and specific description of the technical content of the disclosure in conjunction with the accompanying drawings and specific embodiments.

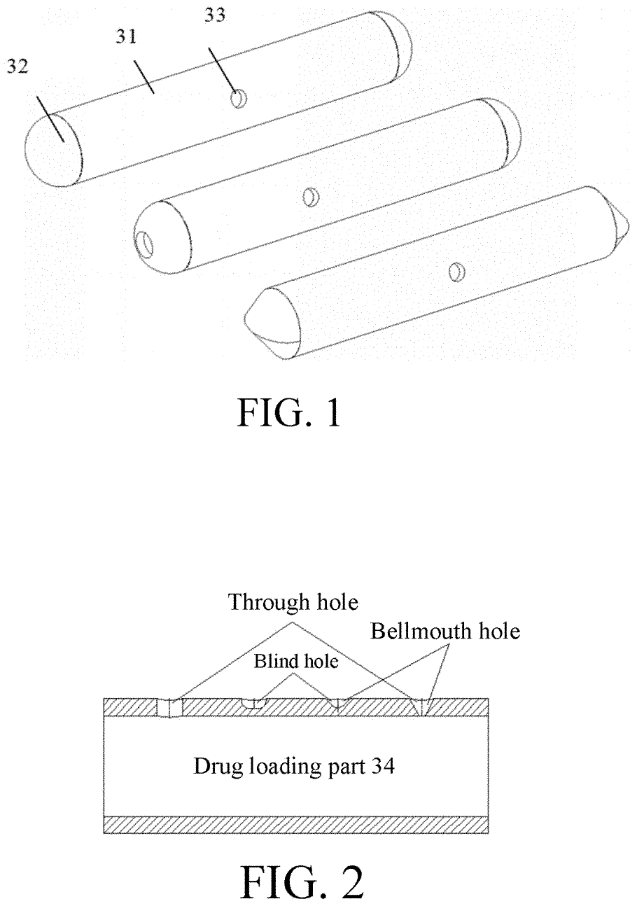

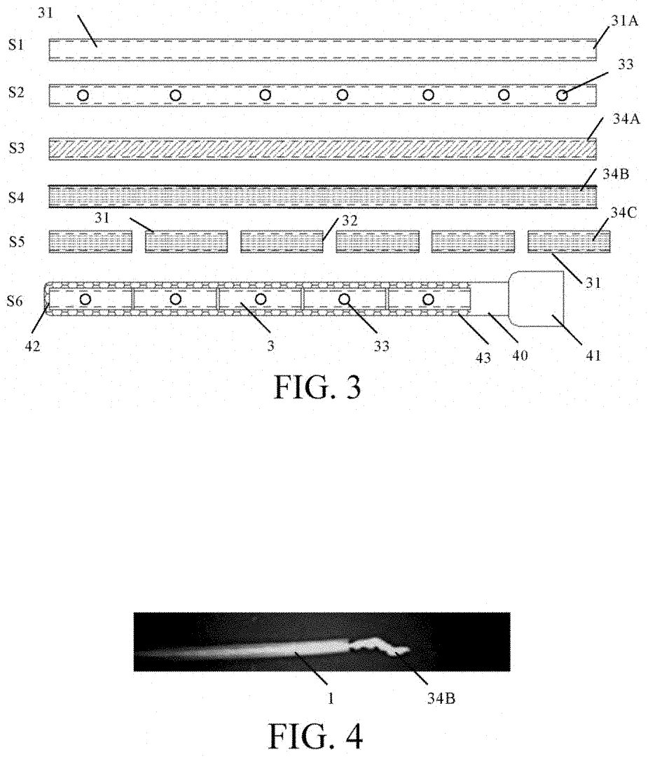

As shown in FIG. 1, a microparticle for drug loading 3 provided by an embodiment of the present disclosure includes a housing 31, a plurality of micro-holes 33 distributed in the housing 31, and a drug loading part 34 (see 34A to 34C in FIG. 3).

As shown in FIGS. 1 and 2, the housing 31 of the microparticle for drug loading 3 is a longitudinal tube made of a medical biodegradable material such as polylactic acid or other conventional medical materials, which has a length set according to practical needs, for example, less than 10 mm, usually in the range of 2-8 mm, and in particular greater than 5 mm and less than 7 mm, and has the same diameter as that of a puncture needle (the same needle core of the puncture needle is needed to push the microparticles from a catheter into the puncture needle). On the one hand, it is conducive to rapid absorption by the human body. On the other hand, because particles are larger than microspheres, the particles can be fixed in a specific location relative to microspheres and avoid being washed away quickly by the blood. The cross-sectional dimensions of the housing 31 are as follows: the inner cavity length is 0.2-0.6 mm, and the outer cavity length is 0.3-0.8 mm. For example, the housing 31 is 6 mm long, the inner cavity size is 0.6 mm, and the outer cavity size is 0.8 mm. Of course, it is understood by those of ordinary skill in the art that the foregoing dimensions can all be adjusted as needed and do not constitute a limit on the present disclosure. For example, microparticles can be longer than 10 mm or even up to several centimeters for implantation into large tumors. The outer diameter of the housing 31 needs to be designed according to the inner diameter of the injection device such as a puncture needle or a catheter.

The housing is sized for implantation into human tissue via a puncture needle. Since the tube diameter specification of conventional puncture needle is 0.8 mm to 2 mm, the outer cavity length of housing 31 should be in the range of 0.5-1.8 mm, too small length will lead to inaccurate pushing when the needle core of the puncture needle is pushed, and too large length will be easily blocked in the puncture needle tube. It is understood by those of ordinary skill in the art that the foregoing dimensions do not constitute a limit on the present disclosure.

As shown in FIG. 2, the cross-section of the housing 31 can be circular, hexagonal, pentagonal or oval, etc. The specific shape is determined by the chosen production process. The material of the housing 31 is preferably PLA, but it can also be other materials with biodegradable properties, especially microsphere carrier materials, such as polycarbonate, polyamino acids, etc. In short, the material of housing 31 can be selected from drug loading materials that are approved by the U.S. Food and Drug Administration (FDA) or National Medical Product Administration (NMPA) or the like, such as polyvinyl alcohol microsphere materials that have been approved by the NMPA, etc.

Both ends of the housing 31 are the ends 32. As shown in FIG. 1, the end 32 can be a hemisphere that closes the port of the housing 31; a hemisphere with a top opening (the diameter of the top opening is less than or equal to the diameter of the micro-hole 33); or a duckbill shape formed by a heat sealing process, and many other shapes. The end 32 may have a through hole to release the drug inside.

The micro-holes 33 are uniformly distributed on the housing 31 and run through the tube wall of the housing 31, as shown in FIGS. 1 and 2. However, it can also be understood by those of ordinary skill in the art that the distribution of the micro-holes 33 can also be not uniform, such as both elongated micro-holes and circular micro-holes distributed on the housing 31; and alternatively, the left half of the housing 31 is distributed with elongated micro-holes, while the right half of the housing 31 is distributed with oval micro-holes, or even forming narrow open slots. Therefore, the micro-holes of this embodiment can have a variety of variations and does not constitute a limit on the present disclosure.

Moreover, a specific surface area of the micro-hole (i.e., for a microparticle for drug loading/drug loading microparticle 3, the area of the micro-hole 33 on the outer surface of the housing 31 as a percentage of the total outer surface area of the housing 31) can be different for different microparticles for drug loading or drug loading microparticles (collectively, microparticles). For example, type A microparticle for drug loading/drug loading microparticle 3 has a specific surface area of the micro-hole of 30% to 40%; and type B microparticle for drug loading/drug loading microparticle 3 has a specific surface area of the micro-hole of 10% to 20%. The specific surface area of the micro-hole increases with degradation time by changing the number of holes, or changing the hole area of the micro-holes in the direction of the wall thickness of housing, and then the drug release speed profile of the microparticles is changed accordingly. Therefore, the release speed profile of the drug can be precisely controlled by designing different specific surface areas of the micro-holes in the direction of the wall thickness of housing via a computer program and creating precise micro-holes in the housing using a laser punching technology.

The aforementioned different specific surface areas of the micro-holes can be obtained by pre-designing in several ways: 1) different number of the micro-holes; 2) different sizes of the micro-holes; 3) micro-hole sizes varying in the wall thickness direction (e.g. bell-mouthed micro-holes); and 4) a combination of the three aforementioned ways.

As shown in FIG. 2, the micro-hole 33 is preferably a cylindrical micro-hole, but can also be a trumpet-shaped micro-hole (on the outer diameter of the housing 31, the diameter of the micro-hole 33 is large; and on the inner diameter of the housing 31, the diameter of the micro-hole 33 is small); or it can be an elongated hole. The shape and size of the micro-hole 33 can be varied and determined by the desired release speed.

Due to the laser punching, the micro-holes formed in the housing strip have a predetermined number and size, even within a predetermined region of the housing. The number and size of the micro-holes are pre-calculated based on the contents inside the drug loading part. Of course, it is also possible to make a series of microparticles for drug loading with different specifications of micro-holes, so that the user can choose the appropriate specification of microparticle for drug loading according to the contents to be loaded. Specifically, for the microparticle for drug loading with an outer diameter of 0.8 mm and an inner diameter of 0.6 mm for drug delivery, the micro-hole 33 can have a size of 0.01-0.4 mm, with a more preferred range of 0.02-0.3 mm, and more preferably 0.02-0.2 mm. Since different contents to be loaded correspond to different preferred micro-hole specifications (size or number), the size of the micro-hole only needs to meet the requirements of the laser punching process as well as the structural requirements of the housing (the micro-hole should not be too large causing the housing to disintegrate easily).

The drug loading part 34 in dry particle form held within the housing 31. The drug loading part 34 may be a hollow area of the housing 31, i.e., an area without any material, for holding the drug liquid or powder. As an alternative, the drug loading part 34 may also be a solid material independent of the housing 31, which is not of integral construction with the housing 31. That is, the drug loading part (34) is a hollow area or a solid having a different material than the housing and the solid can adsorb liquid (liquid such as a medicament or contrast agent).

The material of the drug loading part 34, unlike the material of the housing, is a drug loading material that can absorb liquid and swell into a porous structure with a high drug loading rate, for example, gelatin, or albumin, polylactic acid, polyacrylate, alginate, chitosan, polymethacrylate, etc., which have been shown to be human-usable drug carrier materials, including synthetic biodegradable polymers as well as non-biodegradable polymers. In this embodiment, the drug loading part 34 is described in detail using gelatin as an example, but it does not constitute a limit on the choice of materials in the present disclosure. For example, instead of a gelatin solution, a solution of albumin nanoparticles (referring to prior patent application CN 201310124591.9 for the preparation method) is freeze-dried to prepare albumin nanoparticles, which can also be used as the drug loading part 34. The drug loading part 34 can be prepared from a solution containing polylactic acid or sodium alginate particles in a similar way.

As further detailed in the manufacturing process described below, the freeze-dried gelatin solution is broken into segments (granulated), at which point the drug loading part 34 is a gelatin particle. Then, the microparticle for drug loading 3 is dipped into a drug solution and the drug loading part 34 (gelatin particle) absorbs water and becomes a gelatinous colloid (with drug). Due to the limit from the housing 31, the gelatin will not swell excessively after absorbing water and will only fill the entire interior of the housing 31, and the size of the gelatinous colloid can be controlled.

During the water absorption and swelling of gelatin particles, oil phase components are extruded from the micro-hole 33 and aqueous phase components in the drug solution are absorbed. It can be seen that the drug loading part 34 is a solid having a different material than the housing 31 and the solid can adsorb the liquid entering the housing 31 through the micro-hole 33.

A first method for manufacturing a microparticle for drug loading 3, is described below in connection with FIGS. 3 and 4.

S1: Preparing a housing strip having a predetermined specification

According to the predetermined specification, a strip of housing 31A is selected, washed and sterilized. The housing strip is an elongated tube sealed at one end with an opening at the other end for injection of aqueous gelatin solution.

The predetermined specification means that: individual indicators of the housing strip such as material, size, and cross-sectional shape have been determined in advance. The housing strip can be supplied by the supplier and only selected at the time of manufacture according to predetermined specifications, or can be manufactured in advance. Since this is a conventional technique, it is not repeated herein. A PLA tube of 130 mm was selected in this embodiment.

S2: Injecting the housing with liquid which is a drug loading material and can be freeze-dried to a solid

In this embodiment, the liquid which is a drug loading material and can be freeze-dried to a solid is a predetermined concentration of gelatin solution that becomes granular after freeze-drying and has a high drug loading rate.

An aqueous gelatin solution with a concentration of 3-90% g/ml is injected into the elongated housing 31A, at which point the drug loading part 34 appears as an aqueous gelatin solution 34A in the form of liquid.

The aqueous gelatin solution is formulated in a conventional technique, for example, an aqueous gelatin solution with a concentration of 3%, 4%, or 5% is mixed and stirred using a magnetic stirrer.

S3: Freeze-drying the gelatin solution in the housing under vacuum

The housing strip injected with an aqueous gelatin solution are placed into the vacuum freeze dryer and freeze-dried for 22 h (power-on parameters for chiller: −31.9° C., vacuum pump: −65.1° C., and vacuum gauge: 0.001 Pa) and 68 h (power-on parameters for chiller: −31.9° C., vacuum pump: −65.1° C., and vacuum gauge: 0.001 Pa).

Then, the state of gelatin after freeze-drying and the state of gelatin freeze-dried into gelatin particle 34B are observed using a microscope (16× magnification). It can be observed that after freeze-drying under vacuum for 22 h and 68 h, the flocculent powder formed by gelatin particle are attached to the tube walls or inside the tubes after water sublimation (FIG. 4).

The aqueous gelatin solution can also be kept in the low-temperature vacuum equipment at a temperature of (−60 to −30°) C. for (6 to 10) h and then removed, so that it is freeze-dried into the gelatin particle 34B in the form of flocculent powder.

In the case of albumin nanoparticle solution, as previously described, the albumin nanoparticle solution is freeze-dried. The method for preparing the albumin nanoparticle solution can be referred to the prior patent application CN 201310124591.9 and will not be described herein.

S4: Laser punching

The laser punching is performed on the elongated housing 31A using a laser punching machine, such as the laser punching machine model S-UV-5 from Suzhou Sindway Co., Ltd. The micro-hole 33 is formed by punching in this step. The diameter, shape, specific surface area of hole and the like of the micro-hole 33 are predetermined as described previously. The formation of different micro-holes in the housing strip is achieved by controlling the laser.

As an alternative, it is also possible to adjust step S4 to after step S5 and a laser punching machine for the controlled-release tablet is used to print the housing strip that has been heat-pressed into particles piece by piece.

S5: Heat-pressing into particles and sealing

The elongated housing strip after punching, together with the dried gelatin particles therein, is heat-pressed on a heat sealing machine and sealed at both ends to form a plurality of microparticles for drug loading 3. The process of heat-pressing into particles will result in the duckbill-shaped end 32 in FIG. 1. Other processes can also be used to shear the elongated housing 31A into a segmented housing 31 and sealed at both ends so as to form the microparticles for drug loading 3. At this point the drug loading part 34 becomes the state of small gelatin particles 34C.

S6: Packing into a particle containing tube for sealing and storage

A plurality of microparticles for drug loading 3 are sterilized and then delivered sequentially to the particle containing tube 4 for sealing and storage. The particle containing tube 4 includes a holding chamber 40, a closure end 42, an inlet end 41, and a plurality of perforations 43 distributed within the holding chamber 40. The inner diameter of the inlet end 41 is larger than that of the holding chamber 40 to facilitate the loading of the microparticles for drug loading 3. As shown in FIG. 3, a plurality of microparticles for drug loading 3 are sequentially held inside the holding chamber 40 of the particle containing tube 4, with one end fixed by the closure end 42 and the other end closed by the inlet end 41 (the inlet end 41 is sealed after loading the microparticles for drug loading 3). The tube body is made from materials such as plastic, resin or glass, preferably high-performance polyolefin thermoplastic elastomer (TPE), such as the new TPE material model MT-12051 from Polymax TPE.

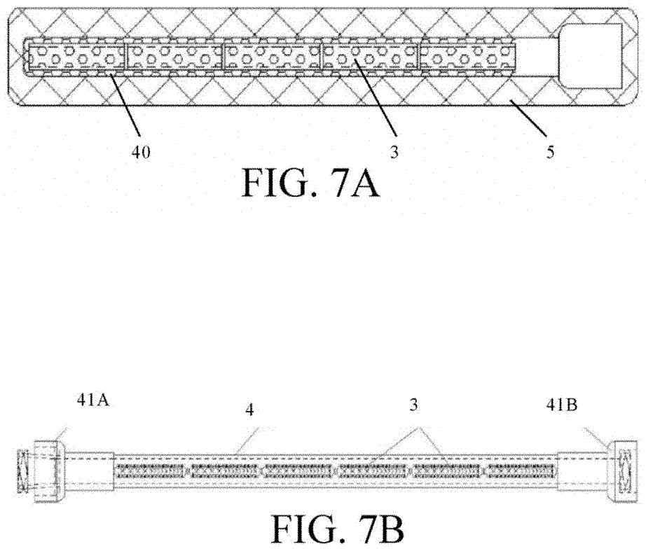

As an alternative, the particle containing tube 4 is sterilized and wrapped inside the outer package 6 under vacuum for easy storage and transportation, etc., as shown in FIGS. 7A and 7B. One end of the particle containing tube 4 (41 in FIG. 7A) is a female luer fitting, or both ends of the particle containing tube 4 (41A/41B in FIG. 7B) are male or female luer fittings, respectively, for connection to an injection needle, puncture needle, or catheter, etc. The connection of a plurality of tubes 1 can be achieved by mating a male end of one of the tube 1 with a female end of the other, thus enabling an increase in the amount of drug (i.e., a continuous supply of drug loading microparticles in a plurality of tubes 1 can be achieved).

The inner diameter of the luer fitting is e.g. 2 cm (only needs to match the outer diameter of the puncture needle or catheter). The particle containing tube 4 is Teflon tube with 2 mm outer diameter and 1 mm inner diameter. As shown in the figure, each particle containing tube 4 stores 5 microparticles for drug loading 3. It is understood by those of ordinary skill in the art that the above quantities or dimensions are illustrative and do not constitute a limit on the present disclosure.

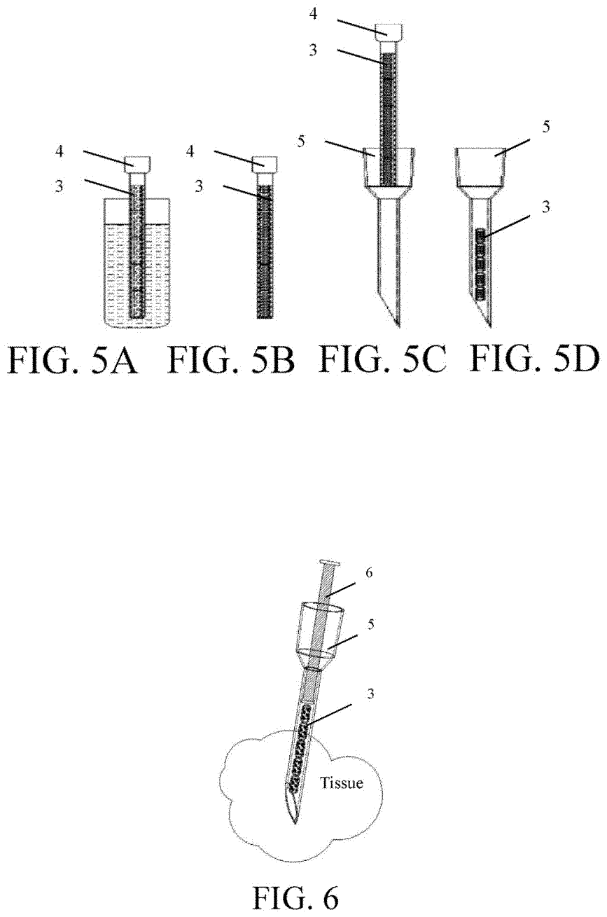

As shown in FIGS. 5A to 5D, before the puncture procedure, the particle containing tube 4 containing microparticles for drug loading 3 is placed in the drug solution. Depending on the factors such as adsorption properties and hydrophilicity of different drugs, after a specific period of time, the drug loading part 34 in the microparticle for drug loading 3 absorbs the drug solution and swells, and the gelatin particle becomes colloidal and fills the housing 31 of the microparticle for drug loading 3, but are protected by the housing 31 from overflowing into the particle containing tube 4 to form the drug loading microparticle. Then, as shown in FIG. 5C, the particle containing tube 4 is inserted into the puncture needle 5 and the drug loading microparticle in the particle containing tube 4 is pushed into the needle tubing of the puncture needle 5 using a pusher (e.g., a flat-tipped apex needle) (as shown in FIG. 5D). Ultimately, as shown in FIG. 6, the body tissue is injected using a puncture needle 5 under the thrust of a puncture needle core 6 (e.g., a flat-tipped apex needle). It is understood by those of ordinary skill in the art that the drug loading microparticles (i.e., microparticles for drug loading after drug loading) can also be injected into the body by means of catheter injection; and the drug loading microparticles are pushed either with a push rod or with a pressurized liquid or gas.

As previously described, the microparticle for drug loading 3 provided by embodiments of the present disclosure is delivered into the body tissue via a puncture needle 5 and is retained within that tissue. The housing 31 of the microparticle for drug loading 3 can remain fixed in a target location within the tissue for a longer period of time due to the large enough size. Compared to nanoparticles or microspheres, the drug within the drug loading part 34 in the microparticle for drug loading 3 which can be fixed at that position will be released continuously, improving the precision of distribution and therapeutic effect.

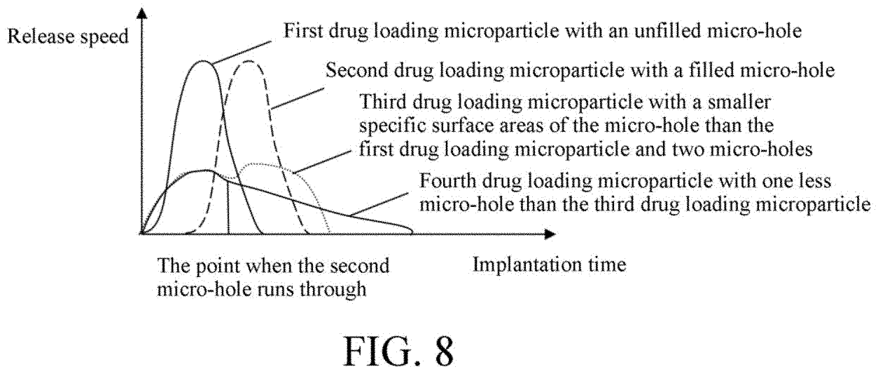

Referring to FIG. 8, the drug release profile of the drug loading part 34 in the microparticle for drug loading 3 can be designed in a variety of shapes by varying a specific surface area of the micro-hole, and by utilizing designs such as fillers to meet different drug release requirements. For example, if the first microparticle in a plurality of microparticles for drug loading 3 has only 1 micro-hole and a specific surface area of the micro-hole greater than that of the fourth microparticle (which also has only 1 micro-hole), the first microparticle is released faster than the fourth microparticle. The only 1 micro-hole of the second microparticle is filled with a slow-dissolving hole filler, after a period of delay, the filler is completely dissolved and the second microparticle exhibits the same release speed profile as the first microparticle, which achieves a delayed release. The third microparticle has 2 micro-holes with one running through the wall thickness of the housing (first micro-hole) and the other not running through the wall thickness of the housing (second micro-hole), but has a surface area of the micro-hole smaller than that of the first microparticle, and thus is released slowly. After the second micro-hole becomes penetrated due to degradation, the release speed is increased and then decreased rapidly. The fourth microparticle differs from the third microparticle only in the absence of the second micro-hole, so that the release speed is continuously decreased after reaching a peak.

In addition, as an alternative, even if a plurality of microparticles for drug loading 3 in the same particle containing tube 4 have the same drug and drug loading part, the microparticles for drug loading 3 each with different release speed profiles can be selected, so that a comprehensive release speed profile can be achieved using different release speed profiles of different microparticles 3. For example, if the first microparticle loaded into the same particle containing tube 4 has a release speed profile shown by the solid line in FIG. 8, the second microparticle (only differs from the first microparticle in that a micro-hole thereof is filled with a slow-dissolving hole filler, and thus after implantation, the drug is not released until the filler dissolves) has a release speed profile shown by the dotted line in FIG. 8, and the drug release profile obtained by implanting such first and second microparticles in the particle containing tube 4 into the body tissue together is the profile of the solid line in FIG. 8 combined with the dotted line in FIG. 8. As a result, the combined profile of the two has release characteristics that are more consistent with drug release requirements than the solid or dotted line in FIG. 8 alone, achieving more precise drug release control.

Furthermore, a plurality of drug loading microparticles implanted by the puncture needle at a time (either implanted one by one or implanted by connecting the puncture needle to the particle containing tube 4 and pushing all drug loading microparticles held in the particle containing tube 4 into the body at a time) can also adjust the combined release profile of all drug loading microparticles through the number of different types of drug loading microparticles to be loaded. For example, assuming that the same puncture needle holds 5 drug loading microparticles, where the number of type A drug loading microparticles 3 with a large specific surface area of the micro-hole is 3 and the number of type B drug loading microparticles 3 with a small specific surface area of the micro-hole is 2. Type A drug loading microparticles with a large specific surface area of the micro-hole have a fast drug release speed; and type B drug loading microparticles have a slow drug release speed. Optionally, type A drug loading microparticles can be injected at one time; and type B drug loading microparticles can be injected at another time to meet the therapeutic needs. The release time can be extended by implanting particles with different specific surface areas of the micro-hole, for example from 14 days to 21 days or even 24 days.

As shown in FIG. 5A, before the doctor performs the procedure, the particle containing tube 4 containing a microparticle for drug loading is placed in the drug solution, and the drug loading part 34 will be attached to the drug in the solution, which can be a radioactive particle or contrast agent (such as a contrast agent containing iodine, barium, etc.), or liquid or suspension such as a solution for conditioning acid-base balance, or an oncotherapy sensitizer, anti-cancer drug, etc. For example, mitomycin, thalidomide, iodine 125 radioactive particles and the like. These drugs, radioactive particles, contrast agents, etc. are collectively referred to as contents. If different drugs are to be used (no adverse reaction after mixing different drugs in the body), the drugs can be made into a mixture solution (cocktail) and added with the microparticle for drug loading 3 (particle containing tube 4) for drug loading.

A plurality of drug loading microparticles implanted by the puncture needle at a time each have different drugs (i.e., the plurality of drug loading microparticles in the particle containing tube contain different drugs), thereby increasing the efficacy of the drug. For example, a first drug loading microparticle is loaded with a first drug and a radioactive particle, a second drug loading microparticle is loaded with a second drug and a collagenase II drug for treating breast cancer, a third drug loading microparticle is loaded with thalidomide, a fourth drug loading microparticle is loaded with mitomycin, and a fifth drug loading microparticle is loaded with a radioactive particle. First, such implantation of different microparticles at a time reduces the number of radioactive particles, since it is possible to locate the five drug loading microparticles without the need for all five drug loading microparticles to be radioactive where the microparticles are arranged sequentially, and the first and last microparticles are radioactive. Second, the amount of collagenase II drugs needed for cancer treatment can be reduced since mitomycin can significantly reduce the resistance of cancer cells. Moreover, thalidomide can disrupt the growth of new blood vessels supplying blood for the cancer cells and improve the efficacy of collagenase II drugs needed for cancer treatment, and thus such drugs are mutually reinforcing drugs. Of course, it is also possible to place only one drug loading microparticle or a plurality of drug loading microparticles with the same drug within the housing.

As an alternation, in the plurality of drug loading microparticles implanted through the puncture needle at a time, the last one loads a procoagulant. After implantation is complete, the last microparticle is implanted into the needle passage of puncture needle. On one hand, the needle passage can be refilled and a physical compression is performed for hemostasis. On the other hand, the procoagulant is released in the needle passage of puncture needle and plays a local hemostatic role. Antibiotics can also be loaded, which is released in the needle passage of puncture needle to prevent needle passage infection. Thus, the present disclosure can prevent the complication of bleeding from the needle passage of puncture needle.

Thus, it can be understood by those of ordinary skill in the art that: forming the drug loading microparticles with different release profiles as described above by using a specific surface area of the micro-hole that changes with degradation time; and combining with the use of loading different contents facilitate both the mutual promotion of a plurality of drugs and the precise control of the drug release speed.

### Second Embodiment

The first embodiment provides microparticles for drug loading without drug loading, and the drug loading operation is performed by the user at the time of use, so that the microparticles for drug loading are loaded to form drug loading microparticles. Provided in this embodiment are drug loading microparticles particles that can be used immediately by the user without the need for a drug loading operation.

<First Method for Manufacturing Drug Loading Microparticles>

S1: Preparing a housing strip having a predetermined specification

Thermoplastic drug controlled release materials such as poly(lactic acid-glycolic acid) (PLGA) or PLA material (polylactic acid, also known as polylactide) are selected. Radioactive particles can also be added to PLA or PLGA so that the housing contains radioactive particles.

The thickness of the housing strip 31A (that is, the thickness of the housing 31) is formed as needed, for example 0.1-0.3 mm, more preferably 0.2 mm.

S2: Preparing drugs for drug loading

The drug that needs to be encapsulated by the housing as the drug loading part is prepared. It is a drug formulated according to the therapeutic needs, either a suspension, emulsion, etc., or a mixture of gaseous, liquid or solid drugs, either water-soluble or oil-soluble. Radioactive particles or drug loading microspheres, or even poly(a-isobutyl alpha cyanoacrylate) (PIBCA) nanoparticles loaded with oligonucleotides, can also be added to the aforementioned drug solution.

S3: Forming particles that encapsulate the drug

In the process of extruding PLA or PLGA, various forms of drugs are injected into their central positions, and the drugs are encapsulated based on the thermoplastic of PLA or PLGA, cooled and formed into a long strip with drugs therein. At this time, the drug solution becomes the drug loading part 34 inside the housing 31, and the PLA or PLGA becomes the housing that encapsulates the drug loading part 34.

S4: Partial thinning the housing

The housing strip 31A is slotted 33 to form the housing strip 31B using a laser punching machine, such as the laser punching machine model S-UV-5 from Suzhou Sindway Co., Ltd. The diameter, shape, distribution position, etc. of the open slot 33 are all predetermined. The open slot 33 is formed on the outer housing of the particles formed in step S3 by controlling the laser energy, pulse time and the like. There is at least one open slot 33 on each particle that does not run through the wall thickness of the housing 31.

S5: Heat sealing into particles

The partially thinned housing strip are made into particles using a heat sealing machine to obtain drug loading microparticles 3 with drug loading part 34 therein.

<Second Method for Manufacturing Drug Loading Microparticles>

S11: Preparing a housing strip having a predetermined specification

The difference between this step and that in the first embodiment is that the housing strip with both ends closed is selected.

S12: Laser punching

The housing strip is punched by using the laser, similar to step S4.

S13: Injecting the housing strip with liquid which is a drug loading material and can be freeze-dried to a solid

This step is similar to S2, excepting that the gelatin solution is injected through the micro-hole formed by punching in step S12. This step including injecting a gelatin solution with a concentration of not less than 50%, for example, a gelatin solution 34A with a concentration of 60-90% g/ml into the housing strip 31A. The surface tension of the highly concentrated gelatin solution will keep the gelatin solution fixed inside the housing strip 31A and will not spill out.

The steps of freeze-drying under vacuum and heat-pressing into particles and sealing are the same as extrusion method.

<Third Method for Manufacturing Drug Loading Microparticles>

S21: Preparing a housing strip having a predetermined specification.

Step S11 in the second method is used as a reference, but the housing strip is closed at both ends.

S22: Laser slotting of the housing strip

Referring to step 13, excepting that at least one open slot running through the wall thickness of the housing strip, and a plurality of open slots not running through the wall thickness of the housing strip (ensuring that there is at least one open slot not running through the wall thickness of the housing strip on each particle) are formed in this step.

S23: Drug loading of the housing strip

Referring to step 12, the liquid drug is injected into the housing strip through open slot running through the wall thickness of the housing strip in step 22. A gelatin solution is preferred.

S24: Vacuum freeze-drying (referring to step 14)

S25: Filling the open slot running through the wall thickness of the housing

The open slot running through the wall thickness of the housing is filled with a slow-dissolving hole filler so that the drug inside the housing strip does not leak out. Moreover, the filler is a water-soluble material that degrades faster than the housing. After such drug loading microparticle is implanted, the drug can be released from the housing, provided that the filler has been dissolved and the open slot in the housing has been exposed, thus achieving the purpose of delayed release.

S26: Heat sealing into particles (referring to step 15)

### Third Embodiment

Unlike the first embodiment, a drug solution containing a preselected drug, such as 50-200 mg of gelatin solution and 10-20 mg of cisplatin (for hepatic artery chemoembolization), is injected into the housing of the microparticle for drug loading in this embodiment. It can also be radioactive particles or drug loading microspheres, etc. It can also be liquid anti-cancer drugs, oncotherapy sensitizers, coagulants, etc. It can also be a mixture of these drugs with radioactive particles, or with drug loading microspheres.

The microparticles for drug loading provided by embodiments of the present disclosure have a large size and can be implanted directly into body tissues without passing through blood vessels, which can improve the precision of the distribution. Moreover, drug loading microparticles with different drugs can be implanted through a single puncture needle injection, thus allowing different drugs to promote each other and improve efficacy. The present disclosure allows the implantation of a plurality of drug loading microparticles with different drug release profiles at a time, which can achieve more precise control of drug release. According to the technical solution provided by the present disclosure, the drug release speed profile can be designed by computer programming, the laser punching can be controlled accordingly to change a specific surface area of the open hole, and then each drug loading microparticle is punched according to the set time by selecting a slow-dissolving hole filler (the open holes not running through are changed as the open holes running through), so that the drug inside the drug loading microparticle can be released according to the ideal release speed profile, thus improving the controlled release accuracy and therapeutic effect, which improves the production and manufacturing of implanted drugs and can increase the production efficiency while improving the efficacy of the drug.

### Fourth Embodiment

Unlike the first embodiment, the microparticle for drug loading 3 in this embodiment shows a hollow structure. That is, the drug loading part 34 within the housing 31 is a hollow area, and the housing 31 is provided with a plurality of micro-holes 33 running through the wall thickness of the housing 31. The contents can be accessed from the outside of the housing 31 through the micro-holes 33 to the drug loading part 34 (i.e., the hollow area of the housing 31). The micro-hole 33 is formed by laser punching and runs through the entire wall thickness of the housing 31.

As an alternative, the micro-hole 33 can also be formed by laser punching, which runs through the entire wall thickness of the housing 31, and the micro-hole 33 is filled with a filler so that the contents within the housing 31 will not flow out. The filler is a water-soluble material that degrades faster than the housing. After such drug loading microparticle is implanted, the drug can be released from the housing, provided that the filler has been dissolved and the open slot in the housing has been exposed, thus achieving the purpose of delayed release.

The microparticle for drug loading is prepared by: 1) preparing a housing strip having a predetermined specification; 2) laser punching to form micro-holes 33 in the housing strip; and 3) heat-pressing into particles and sealing. A more optimized preparation method includes: 1) preparing a housing strip having a predetermined specification; 2) laser punching to form micro-holes having predetermined numbers and sizes in the housing strip; 3) heat-pressing into particles and sealing; 4) filling the micro-holes with a predetermined filler; and 5) cleaning the excess filler and drying.

### Fifth Embodiment

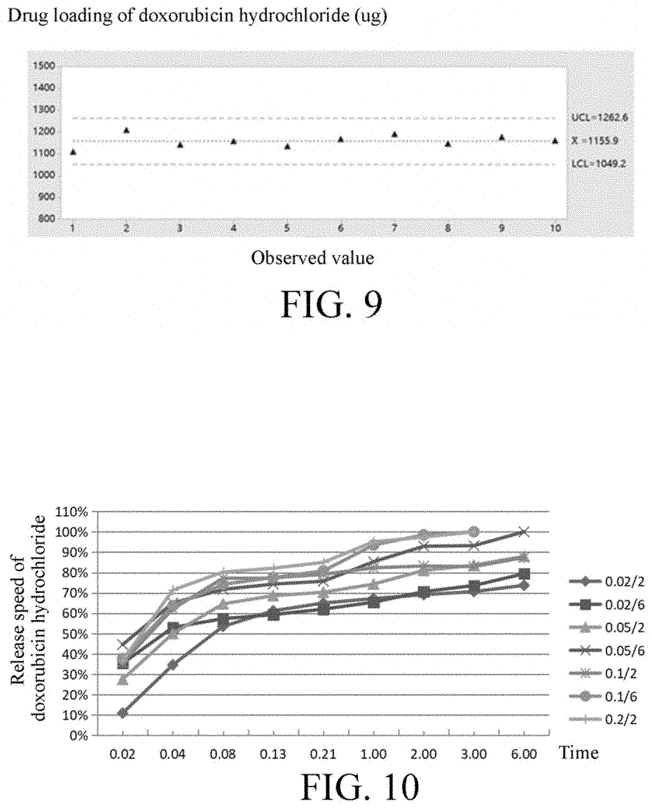

The following is an example of a specific type of microparticle for drug loading. As shown in FIG. 9, the housing 31 for the microparticle for drug loading is 10 mm long, with an outer diameter of 0.8 mm and an inner diameter of 0.6 mm; and the drug loading part 34 is a hollow area. The preloaded drug solution in the drug loading part 34 of the microparticle for drug loading is a mixture obtained by adding 700 mg of adriamycin hydrochloride to 1 ml of DMSO solvent. After several observations, the average drug loading of adriamycin hydrochloride was around 1.16 mg in a single microparticle for drug loading having the aforementioned specification.

The manufacturing method for this particular model is as follows. First, the mix nylon, polytetrafluoroethylene, polylactic acid (PLA), PLA-PEG and polyester elastomer type polymer material tube (PLA tube was used in this embodiment) were washed and cut using a sealing machine. The sealing temperature was 105° C. A laser punching machine was then used to punch. The number, location and size of the holes were pre-designed according to actual needs. Finally, washing, drying and packing were performed. When in use, the microparticle for drug loading was taken out from the package and injected into the prepared liquid containing the contents for drug loading.

FIG. 10 is the release profile of doxorubicin hydrochloride contained in the microparticle for drug loading in normal saline. It can be seen that different diameters and numbers of the holes correspond to different release characteristics. In Table 1 below, “0.02/2” means that the microparticle for drug loading is 10 mm long, with an outer diameter of 0.8 mm and an inner diameter of 0.6 mm, and each microparticle for drug loading has 2 open holes of 0.02 mm in diameter (all holes are through holes). It is known to those of ordinary skill in the art that products with different models can be designed depending on different diameters/numbers of pores to meet the needs of different conditions.

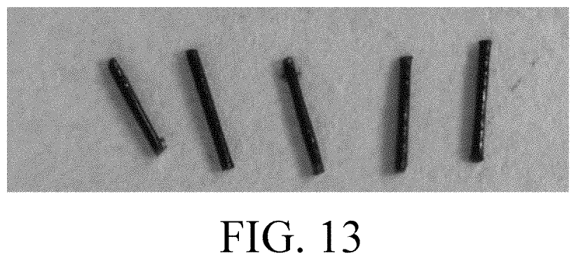

After the microparticle for drug loading was loaded according to the above method, it became drug loading microparticle (as shown in FIG. 13) and was tested for release rate.

In Tables 1 and 2 below, the 3 h release rate represents the percentage of drug solution that has been released into normal saline as measured by placing the drug loading microparticle in normal saline for 3 h. The 3 d release rate represents the percentage of drug solution that has been released into normal saline as measured by placing the drug loading microparticle in normal saline for 3 days. The recovery (6d) represents the ratio of the drug solution measured in the normal saline upon immersing the drug loading microparticle in normal saline for 6*24 h to the drug solution pre-loaded in the drug loading microparticle. It can be seen from the table below that the 0.1 mm/6 sized drug loading microparticle and the 0.2 mm/2 sized drug loading microparticle are completely released in 3 days (3d release rate: 100%) due to largest diameters thereof. The 0.02 mm/2 sized drug loading microparticle has a 3d release rate of 69.4% and then releases slowly until day 6 when the recovery rate only reaches 74%. In other words, 26% of the drug is still not released. It can be seen that the smaller the diameter of holes and the smaller the number of holes, the slower the release rate of the drug loading microparticle. Thus, different drug release speed can be adjusted by designing the diameter and/or number of holes to meet different clinical needs.

TABLE 1
Example of diameter and/or number of holes design

| | |
|---|---|
| Diameter | Number |

| | | | | | |
|---|---|---|---|---|---|
| 0.02 | mm | 2 | 4 | 6 | 8 |
| 0.05 | mm | 2 | 4 | 6 | 8 |
| 0.1 | mm | 2 | 4 | 6 | 8 |
| 0.2 | mm | 2 | 4 | 6 | 8 |

TABLE 2
Drug release rates of drug loading microparticles having various specifications

| | 0.02/2 | 0.02/6 | 0.05/2 | 0.05/6 | 0.1/2 | 0.1/6 | 0.2/2 |
|---|---|---|---|---|---|---|---|
| 3 h Release rate | 57.4% | 60.5% | 66.7% | 74.4% | 78.5% | 72.8% | 79.8% |
| 3 d Release rate | 69.4% | 74.2% | 80.3% | 92.1% | 83.4% | 100% | 100% |
| Recovery (6 d) | 74% | 80% | 84.5% | 99.1% | 88.6% | 100% | 100% |

To evaluate the safety and efficacy of the microparticles, nude mouse experiments were performed. An animal model of liver cancer in nude mice was prepared, and a microparticle for drug loading with adriamycin hydrochloride was implanted in the tumor to observe the inhibitory effect of the drug in the microparticle for drug loading on tumor growth, and the procedure and effect of the experiment were as follows.

I. Experimental Animals

19 healthy male BALB/c nude mice, aged 4-5 weeks, weighing 16-20 g, were selected.

II. Experimental Method

2.1 Preparation of Mouse Animal Model

When the average diameter of the tumor reached about 8 mm, nude mice with good tumor growth, no spontaneous hemorrhage and necrosis, and no infection lesions around the tumor were selected. The tumor was removed aseptically, cut into 1 mm³ in size and subcutaneously inoculated into the forelimb axilla of nude mice. About 1 week later, the model was successfully prepared when the tumor diameter >3 mm was visible to the naked eye, and the experiment was started when the tumor diameter reached 10 mm±2 mm.

2.2 Implantation Scheme

20 nude mice in the model were implanted based on the following schemes.

(1) Control group (Group A): 10 blank control nude mice were selected, and 2 microparticles for drug loading (loading DMSO) without drug were implanted into the tumor of each nude mouse.

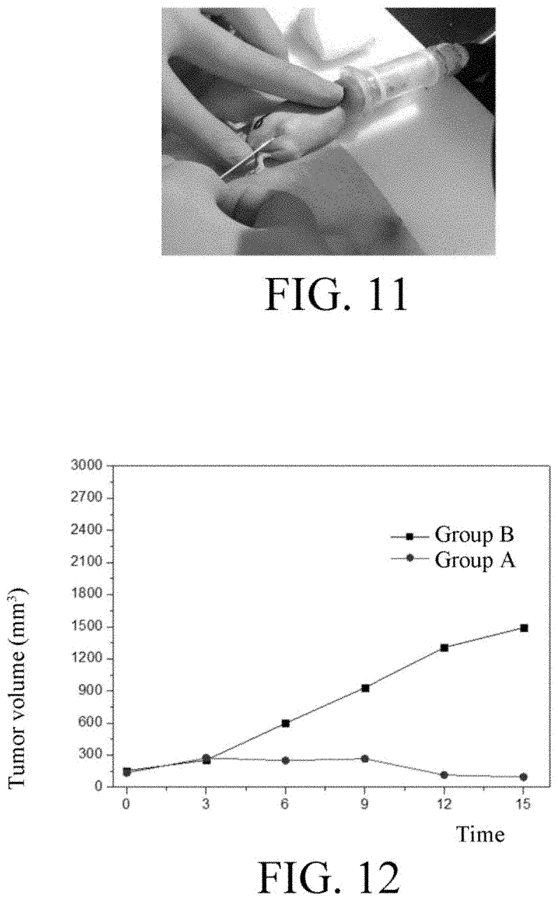

(2) Treatment group (Group B): the drug loading microparticles loading adriamycin hydrochloride API were implanted intratumorally in 10 nude mice at 1 microparticle per mouse, and an 18G puncture needle was used for implantation (as shown in FIG. 11).

2.3 Observation Indicators

(1) Before and after the experiment, the tumor formation and general growth of nude mice in each group should be observed.

(2) The body weight of the animals was recorded before the drug loading microparticles were implanted. The body weight of the animals was measured at intervals of 3d/6d/9d/12d/15d/18d/21d after administration. Changes in animal body weight over time were recorded.

(3) The longest diameter (a) and widest diameter (b) of the tumor were measured with an electronic vernier caliper before the drug loading microparticles were implanted, and the tumor volume was calculated according to the formula V(cm³)=ab²/2. Tumor diameter was measured at different times (3d/6d/9d/12d/15d/18d/21d) after administration and tumor volume was calculated. Tumor growth was recorded, and change in the tumor volume over time was plotted.

(4) The experiment was finished 20 days after the drug loading microparticles were implanted. All animals were executed by the cervical dislocation method, the tumors were removed and weighed, and the tumor suppression rate (TSR) was calculated according to the formula. TSR=(1−Wt/Wc)×100%, where We represents the mean weight of tumor in group A and Wt represents the mean weight in group B.

(5) Pathological sections were made at the end of the test to observe the necrosis of tumor cells and the infiltration of inflammatory cells.

III. Experimental Data Collection and Analysis

The measured data were collected, change in the tumor volume over time was plotted, and the tumor suppression rate of the experimental group was calculated.

The following table provides the tumor changes in the nude mice of the aforementioned group A and group B.

TABLE 3
Mean values of tumor volume changes in nude mice of
each group before and after administration (mm³)

| | | Before | | | | | |
|---|---|---|---|---|---|---|---|
| | | treatment | After treatment (day) | | | | |
| Group | Number | (0 d) | 3 d | 6 d | 9 d | 12 d | 15 d |
| Group A | 10 | 154.31 | 253.51 | 598.03 | 931.03 | 1306.74 | 1490.32 |
| Group B | 10 | 135.20 | 273.78 | 250.01 | 266.18 | 115.11 | 95.27 |

It can be seen from the above table that group B showed a significant difference in tumor volume compared to group A on day 6 after treatment. As shown in FIG. 12, tumor growth in group B showed tumor shrinkage and decreased growth curve. Group A grew faster and had a steeper curve. The tumor volume in group B was significantly smaller than that in group A, indicating that the drug loading microparticle can significantly increase the local drug concentration and exert the effect of inhibiting tumor growth after implantation.

It is also found in this study that the nude mice implanted with drug loading microparticles showed no mortality and smooth and safe drug release after implantation.

TABLE 4
Tumor weight and tumor suppression rate

| | | | Tumor |
|---|---|---|---|
| | | Tumor weight | suppression |
| Group | Number | (g) | rate |
| Group A (W_{c}) | 10 | 0.811 | — |
| Group B (Wₜ) | 10 | 0.177 | 78.2% |

The test was finished 15 days after implantation. All animals were executed by the cervical dislocation method, the tumors were removed and weighed, and the tumor suppression rate (TSR) was calculated according to the formula. TSR=(1−Wt/Wc)×100%, where Wt represents the average tumor weight of group B, and We represents the average tumor weight of group A. It can be seen that after implanting the drug loading microparticles, the tumor suppression rate can reach 78.2%, and the drug treatment effect is obvious.

The microparticle for drug loading, the drug loading microparticle, the particle containing tube, and the implantation system for the microparticle provided by the present disclosure have been described in detail above. For those of ordinary skill in the art, any obvious changes made to the present disclosure without departing from the essential content thereof will constitute an infringement on the patent right of the disclosure and will entail corresponding legal liability.

1. A microparticle for drug loading used for being implanted into body tissues by means of a puncture needle (5) comprising a housing (31) and a drug loading part (34) located inside the housing, wherein
the housing (31) is provided with at least one micro-hole (33) running through the wall thickness of the housing (31), and
the drug loading part (34) is located inside the housing (31) and used for loading drugs.

2. The microparticle for drug loading according to claim 1, wherein
the housing (31) is a biodegradable material and a specific surface area of the micro-hole of the housing (31) varies with the degradation time.

3. The microparticle for drug loading according to claim 1, wherein
there are a plurality of micro-holes (33) and at least one of the plurality of micro-holes does not run through the wall thickness of the housing (31); or the micro-hole (33) running through the wall thickness of the housing (31) is filled with a slow-dissolving hole filler.

4. The microparticle for drug loading according to claim 1, wherein
the micro-hole (33) has a predetermined number, size, location or area that varies in the wall thickness direction.

5. The microparticle for drug loading according to claim 1, wherein
the drug loading part (34) is a hollow area, or the drug loading part (34) is a solid having a different material than the housing and the solid can adsorb liquid entering the housing (31) through the micro-hole (33).

6. A drug loading microparticle used for being implanted into body tissues by means of a puncture needle (5) comprising a housing (31) and a drug loading part (34) located inside the housing (31), wherein
the housing (31) is provided with at least one micro-hole (33) running through the wall thickness of the housing (31),
the drug loading part (34) is located inside the housing (31) and has loaded contents, and
the contents are biocompatible and dissolvable into the body tissues outside the housing (31).

7. The drug loading microparticle according to claim 6, wherein
the housing (31) is provided with at least one micro-hole (33) which does not run through the wall thickness of the housing (31) or at least one micro-hole (33) which is filled with a slow-dissolving, hole filler.

8. The drug loading microparticle according to claim 6, wherein
the housing of the drug loading microparticle loads a drug and/or a contrast agent, the drug being a drug different from that in the drug loading part (34).

9-11. (canceled)

12. An implantation system for implanting a drug loading microparticle, comprising a puncture needle (5) or a catheter, and further comprising a particle containing tube (4) which is used for holding a plurality of microparticles for drug loading or drug loading microparticles.

13. The implantation system according to claim 12, wherein
the particle containing tube (4) has a plurality of drug loading microparticles, wherein at least two of the drug loading microparticles contain different contents.

14. The implantation system according to claim 12, wherein
the last one in the plurality of drug loading microparticles loads a procoagulant, a contrast agent or a radioactive particle.

15. The implantation system according to claim 12, wherein
at least two housings of the microparticles for drug loading/drug loading microparticles have different specific surface areas of micro-holes.

16. A therapeutic and/or diagnostic method comprising:
(i) selecting a microparticle of claim 1 based on a drug release speed profile;
(ii) loading a drug or drugs in the drug loading part (34) of the microparticle through the hole(s);
(iii) implanting the microparticle loaded with the drug(s) into a body tissue by means of a puncture needle (5) or a catheter; and
(iv) releasing the loaded drug(s) through the hole(s) into the body tissue to implement a therapeutic and/or diagnostic method.
